(19) United States
(12) Patent Application Publication
(10) Pub. No.: US 2008/0027452 A1
(43) Pub. Date: Jan. 31, 2008

(54) SURGICAL INSTRUMENT

(76) Inventors: Garrett Sheffer, Hoboken, NJ (US); David Wycliffe Murray, Cuddesdon (GB); Edward Alexander Reeves, Dursley (GB); William Keith Thomas, Chippenham (GB)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 11/750,641

(22) Filed: May 18, 2007

Related U.S. Application Data
(63) Continuation-in-part of application No. 11/248,729, filed on Oct. 12, 2005.

(30) Foreign Application Priority Data
Dec. 6, 2004 (GB) GB426767.0

(51) Int. Cl. A61F 2/46

(52) U.S. Cl. 606/89

(57) ABSTRACT

A surgical instrument including base member adapted to be attached to a bone, a surgical device coupled to the base member and a marker removably attached to the base. The device can include guide, for instance a planar cutting surface for guiding a blade or a circular aperture for guiding a drill.

### RELATED APPLICATIONS

This application is a continuation-in-part of and claims the benefit of U.S. patent application Ser. No. 11/248,729, filed Oct. 12, 2005, entitled “SURGICAL INSTRUMENT”, which in turn claims priority to GB 426767.0, filed Dec. 6, 2004, the entireties of which are herein incorporated by reference.

### FIELD OF INVENTION

This invention relates to a surgical instrument and particularly but not exclusively to an instrument for use in surgery employing surgical navigation.

### BACKGROUND

During most types of orthopedic surgery it is necessary to resect specific areas of bone. The precision of resection is particularly important during joint replacement surgery, where it is necessary to make a cut or drill a hole in a precise location and orientation relative to a patient's bone. Traditional methods use mechanical tools to reference off a remote area of bone in order to determine the correct position in which to cut or drill. However, recent advances have produced an alternative to physical referencing in the form of navigated surgery.

In, for example, a knee replacement operation, a permanent marker, comprising an array of three reflective devices or light emitting diodes, is attached to the femur through an incision remote from the knee area. Two cameras are then used to capture the position of the marker, and hence the bone, in space. A probe, comprising a second marker, is then touched to specific areas of the femur, in order to capture each position in space relative to the first, fixed marker. In this way, a digital image of the surface of the bone is created. This image is displayed to the surgeon on a computer screen. During the subsequent operation, the first, fixed marker and cameras remain in place in order to provide a link between the actual bone and the digital image. Any physical movement of the bone is thus reflected on the computer screen. The computer is able to calculate, from the image of the bone, exactly where a cut or hole should be made and hence to indicate on the screen the required position of a cutting or drill guide. The appropriate guide is provided with a marker to enable the surgeon to observe the position of the guide relative to the bone on the computer screen. When the image of the guide is in the required position relative to the bone as viewed on the screen, the guide is fixed in place and the cut is made. A similar procedure is followed for resection of the tibia.

Using navigated surgery, it is possible to achieve greater accuracy in the cutting and drilling of bone. However, the procedures can be difficult to implement. Although the required position of the guide relative to the bone is indicated on the screen, in practice, it is extremely difficult to attach the guide in precisely the right position. The attachment procedure requires the drilling of holes through the bone into which bone screws are inserted to hold the guide in place. Once the holes are drilled, further, fine adjustment of the position of the guide is often not possible. Exact matching of the position and orientation of the guide with the ideal position indicated on the screen is therefore extremely difficult.

A further disadvantage of the procedure is the need for at least one extra incision in the leg, spaced from the operative incision, in order to attach a fixed marker to a bone. This extra incision increases the duration of the patient's post operation recovery time. The provision of the fixed marker also results in additional bone damage, caused by the screws that hold the marker in place.

### SUMMARY OF INVENTION

According to one embodiment of the present invention there is provided a surgical instrument for use with a surgical navigation system. The surgical instrument includes a base adapted to be attached to a bone and having a first marker connected thereto. The first marker can be trackable by the surgical navigation system. The at least two surgical devices can be removably, adjustably and interchangeably coupled to the base member.

According to another embodiment of the present invention there is provided surgical apparatus including a base adapted to be coupled to a bone of a patient, a first reference array coupled to the base, a tool guide adjustably and removably coupled to the base, and an adjustment mechanism, coupled to one of the base and the tool guide. The adjustment mechanism includes a first positioner and a second positioner. The first positioner can be adapted to move the tool guide relative to the first reference array in a first direction. The second positioner can be adapted to move the tool guide in a second direction different than the first direction.

Pursuant to another aspect of the present invention, there is provided a method of cutting a bone during a surgery aided by a surgical navigation system. The method includes the steps of mounting a base member having a reference array to a bone, attaching a cutting guide to the reference array and then positioning the cutting guide using a tracked instrument, fixing the position of the cutting guide relative to the base member, and resecting the bone.

### BRIEF DESCRIPTION OF DRAWINGS

The invention will now be described by way of example only with reference to the accompanying drawings, in which:

FIG. 1 is a diagrammatic perspective view of a first embodiment of a surgical instrument in accordance with the invention.

FIG. 2 a plan view of the surgical instrument of FIG. 1 attached to the upper part of a tibia for performing a partial resection of the tibial plateau.

FIG. 3 is a diagrammatic plan view, partly in cross section, of a second embodiment of a surgical instrument in accordance with the invention.

FIG. 4 is a plan view of the surgical instrument shown in FIG. 3 attached to a femur for drilling a hone in a femoral condyle.

FIG. 5 is a perspective view of another embodiment of a surgical instrument in accordance with the present invention.

FIG. 6 is a perspective view of another embodiment of a surgical instrument in accordance with the present invention.

FIG. 7 is a diagramatic sectional view of quick disconnect fitting of FIGS. 5 and 6.

### DETAILED DESCRIPTION

Referring firstly to FIG. 1, a surgical instrument is indicated generally at 10. The surgical instrument 10 comprises a base member 12 and a cutting guide 14, adjustably connected together by means of a ball and socket joint 16. A planar guiding surface 15 is provided on the cutting guide 14, which, in use, guides a reciprocating saw or other cutting tool. A ball 18, of the joint 16, is mounted at the end of a stem 20, which is slidably received in an aperture 22 in the base member 12. A locking means 24, for example, a locking screw, is provided in the base member 12 for locking the position of the ball 18 and stem 20 relative to the base member 12.

The ball 18 is received in a socket 26 of the joint 16, provided in the cutting guide 14. A second locking means 28 is provided on the cutting guide 14, which locks the cutting guide relative to the ball 18 and stem 20. The position of the cutting guide 14 can therefore be locked relative to the position of the base member 12 by means of the first and second locking means 24,28 acting on the stem 20 and ball 18.

First and second apertures 30, 32 are provided through the base member 12, through which screws may be driven in order to secure the surgical instrument to a bone. A first marker 34 is removably attached to the base member 12, and a second marker 36 is removably attached to the cutting guide 14. The markers are those known in the art of digitised operative treatment or navigated surgery as arrays, and each comprises three spatially positioned light emitting diodes or reflectors 38, which can be identified by a camera or other suitable identifying means.

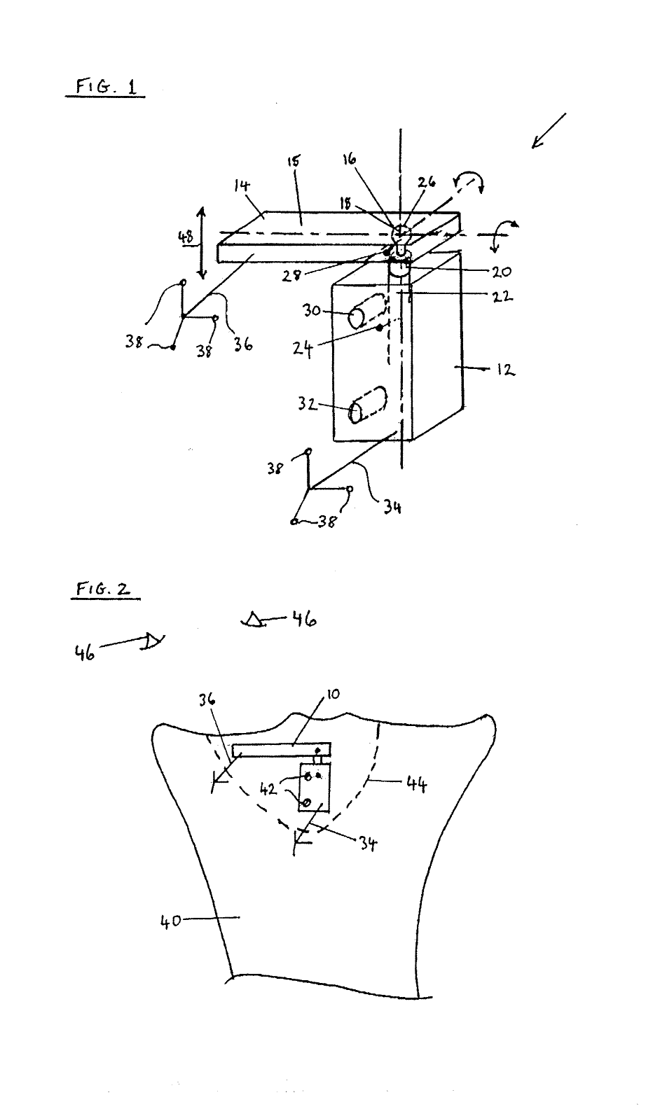

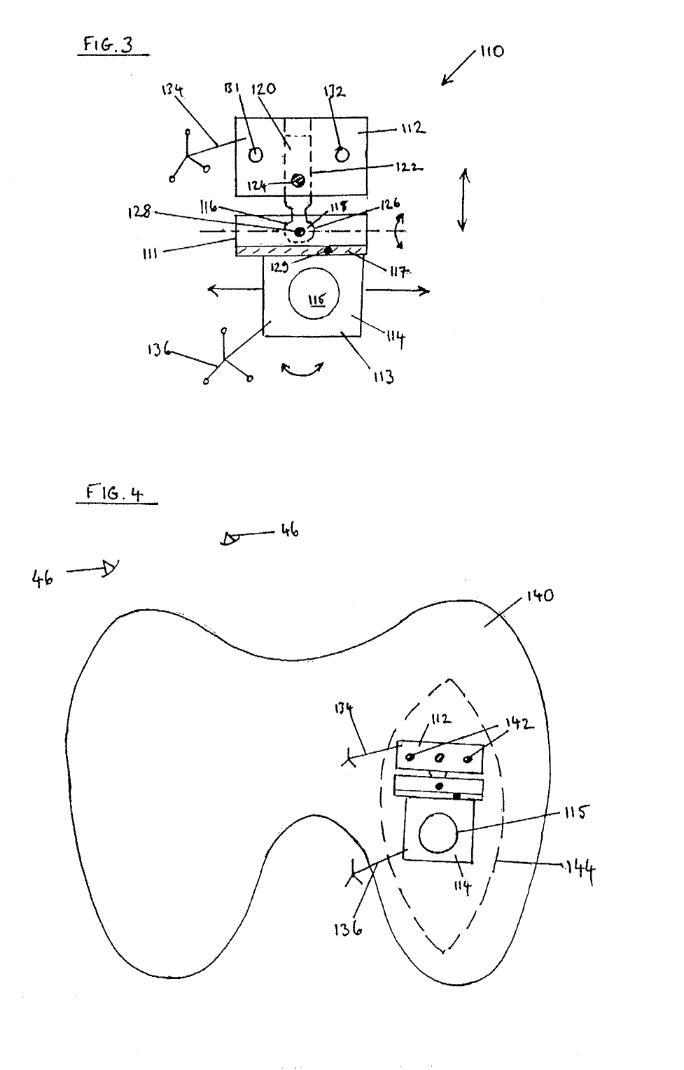

Referring now to FIG. 2, a standard surgical approach has been carried out for performing a partial knee replacement operation and the osteophytes have been removed. The instrument 10 is shown attached to a tibia 40 by means of a pair of screws 42, which are fastened to the tibia through the first and second apertures 30,32. The instrument is positioned against the tibia through a small incision in a patient's knee, which is indicated in dotted outline at 44. The screws are inserted into the tibia in the region of the tibial tubercle. At this point in the operation, the arrays 34,36 are removed from the instrument 10. A pair of cameras 46 capable of detecting the position of the arrays 34, 36 is directed towards the operative area. The cameras 46 are linked to a computer and computer screen (not shown) in a known configuration.

The first array 34 is attached to the base member, which is attached to the tibia 40 and key points around the tibia, are digitized in known manner, by means of a further array attached to a probe (not shown). These points typically include the medial and lateral malleolae; the centre of the tibial plateau; the surface of the posterior part of the medial tibial plateau (with the knee in maximal flexion) and the lateral side of the medial femoral condyle (with the knee in 90° flexion). Then, with the knee in full extension the patient's leg is moved around in order to identify the femoral head. The computer then determines the desired position of the saw cut across the top of the tibia 40. Typically, the plane of the cut will be perpendicular to the tibial axis in the coronal plane and sloping 7° in the saggital plane and 8 millimetres below the normal cartilage posteriorly, although the position of the cut may be adapted to suit a patient's anatomy.

The second array 36 is then attached to the cutting guide 14. The cutting guide 14 is then adjusted until the planar guiding surface 15 is in the correct orientation, which is determined by the surgeon with reference to the computer screen, where a digitised image of the tibia 40, guide 10 and ideal cut position are displayed. In order to adjust the instrument 10, the locking means 24, 28 are released. This allows the cutting guide 14 to move relative to the base member 12, firstly in the direction of the axis of the stem 20, as indicated by arrow 48, and secondly in rotatational directions about perpendicular axes of the ball and socket joint 16 as indicated by the arrows 50,52. When the planar guiding surface 15 is correctly positioned, the locking means 24, 28 are engaged to lock the cutting guide 14 in position relative to the base member 12 and the bone 40, to which the base member is attached. The necessary cut or cuts can then be made and the instrument removed by releasing the screws 42.

Optionally, the cutting guide 14 may have a further planar guiding surface (not shown) positioned substantially perpendicular to the planar guiding surface 15, for guiding a saw or other cutting implement in a vertical cut.

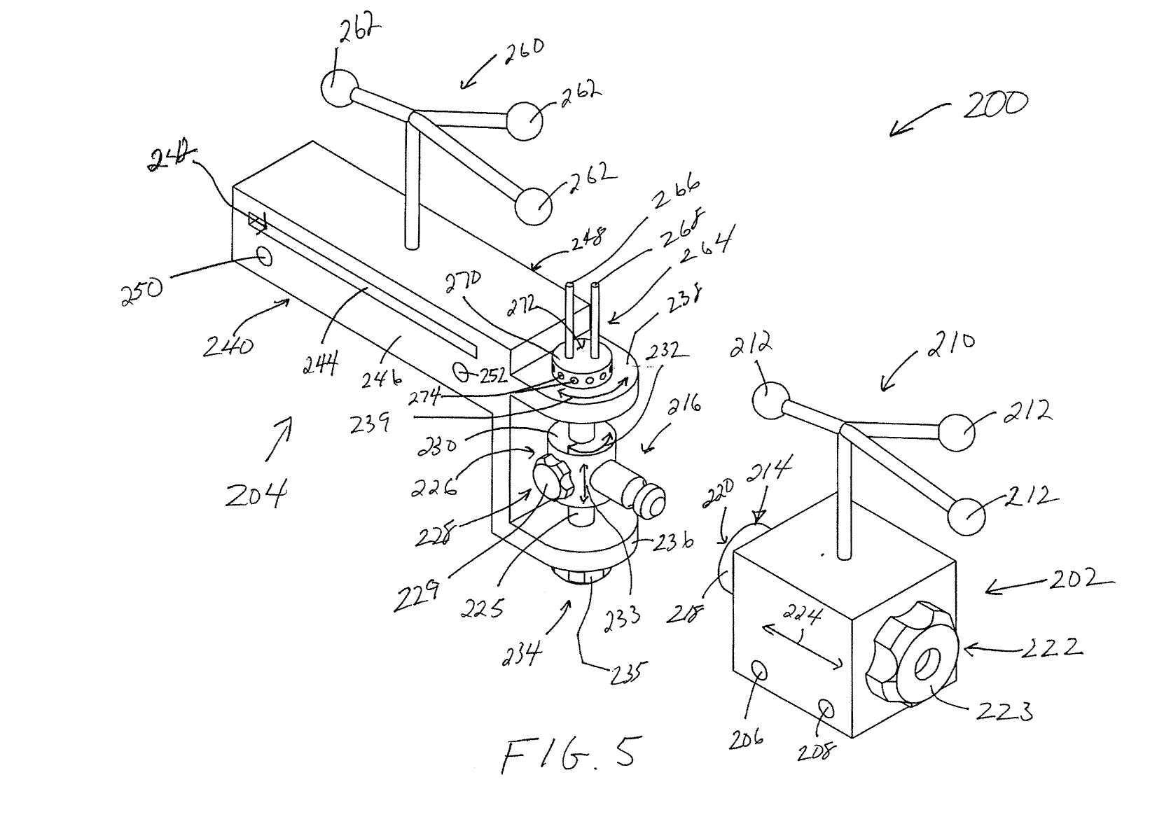

Referring now to FIG. 3, a second embodiment of a surgical instrument is indicated generally at 110. The surgical instrument 110 comprises a base member 112 and a cutting guide 114, adjustably connected together by means of a ball and socket joint 116. The cutting guide 114 is provided in first and second parts 111,113. A circular hole 115 is provided through second part 113 of the cutting guide 114, which, in use, guides a drill, brooch or other cutting implement. A ball 118 of the joint 116 is mounted at the end of a stem 120, which is slidably received in an aperture 122 in the base member 112. A locking means 124, for example, a locking screw, is provided in the base member 112 for locking the position of the ball 118 and stem 120 relative to the base member 112.

The ball 118 is received in a socket 126 provided in the first part 111 of the cutting guide 14. A second locking means 128 is provided on the first part of the cutting guide 114, which locks the cutting guide relative to the ball 118 and stem 120. However, in this embodiment, a further degree of adjustability is provided by a sliding joint 117, which may be a dovetail slide, or any suitable sliding joint, provided between the first and second parts 111,113 of the cutting guide 114. A third locking means 129 is provided in one of the parts 111,113 of the cutting guide 114 for locking the position of the first part 111 relative to the second part 113 of the cutting guide. In this manner, the position of the cutting guide 114 can be locked relative to the position of the base member 112.

As in the first embodiment, first and second apertures 131, 132 are provided through the base member 112, through which screws may be driven in order to secure the surgical instrument to a bone. A first marker 134 is removably attached to the base member 112, and a second marker 136 is removably attached to the second part 113 of the cutting guide 114. The markers are those known in the art of digitized operative treatment as arrays as described previously.

The surgical instrument 110 is shown in use in FIG. 4 during a partial knee replacement operation. The instrument is attached to a femur 140, by means of a pair of screws 142, which pass through the apertures 131, 132. The instrument 110, without the markers 134, 136, is positioned against the femur 140 through an incision, indicated in dotted outline at 144. The operating arrangement is as described with regard to the first embodiment, with cameras 46 linked to a computer and computer screen (not shown) in a known configuration.

The first marker or array 134, is attached to the base member 112, which is attached to the femur 140 and a series of points and planes are digitized in known manner, by means of a further array attached to a probe (not shown). In particular, the patient's leg is moved around in order to determine the longitudinal axis of the femur and the position of the centre of the femoral head. The patient's knee is angled at 90°, and the largest size possible of tibial measurement guide block (not shown) is placed in the flexion gap. The block is pushed against a vertical cut of the tibia, and a further array is attached to the block. The plane defined by the upper surface and the lateral surface of the block are captured by the cameras and computer. These planes define the vertical cut, the orientation of the horizontal cut and the position of the most posterior part of the femoral condyle.

The computer then determines the position of the hole to be drilled in the femur 140. This hole should point directly towards the centre of the femoral head. The distance of the axis of the hole above the posterior part of the femoral condyle is equal to the radius of the femoral component. The distance that the hole is offset from the plane of the vertical cut of the tibia, which is perpendicular to the horizontal cut is equal to half of the width of a meniscal bearing component plus 2.5 mm.

The second array 136 is then attached to the cutting guide 114, and the guide adjusted until it is in the correct orientation, which is determined by the surgeon with reference to the computer screen, where a digitized image is displayed. Once the guide 114 is locked in position by the locking means 124, 128, 129, the arrays 134, 136 are removed from the instrument 110 and the hole drilled in the femur, the drilling tool being guided in the hole 116 provided through the second part 113 of the cutting guide 114. The instrument 110 is then removed by removal of the screws 142.

Once removed, a posterior femoral saw guide (not shown) is inserted into the drilled hole in the femur 140. With the knee at 90° flexion and a valgus load applied to the tibia, the saw guide is aligned approximately parallel to the tibia. A pin is inserted in the upper hole of the guide and the posterior saw cut made. The saw guide is then removed. The operation is then completed in usual manner.

The embodiments 10, 110 of the surgical instrument shown, are typical arrangements of a surgical instrument within the scope of the claims, but it will be appreciated that other arrangements are possible which provide the required adjustability between the base members 12, 112 and the guide 14, 114 and fall within the scope of the claims.

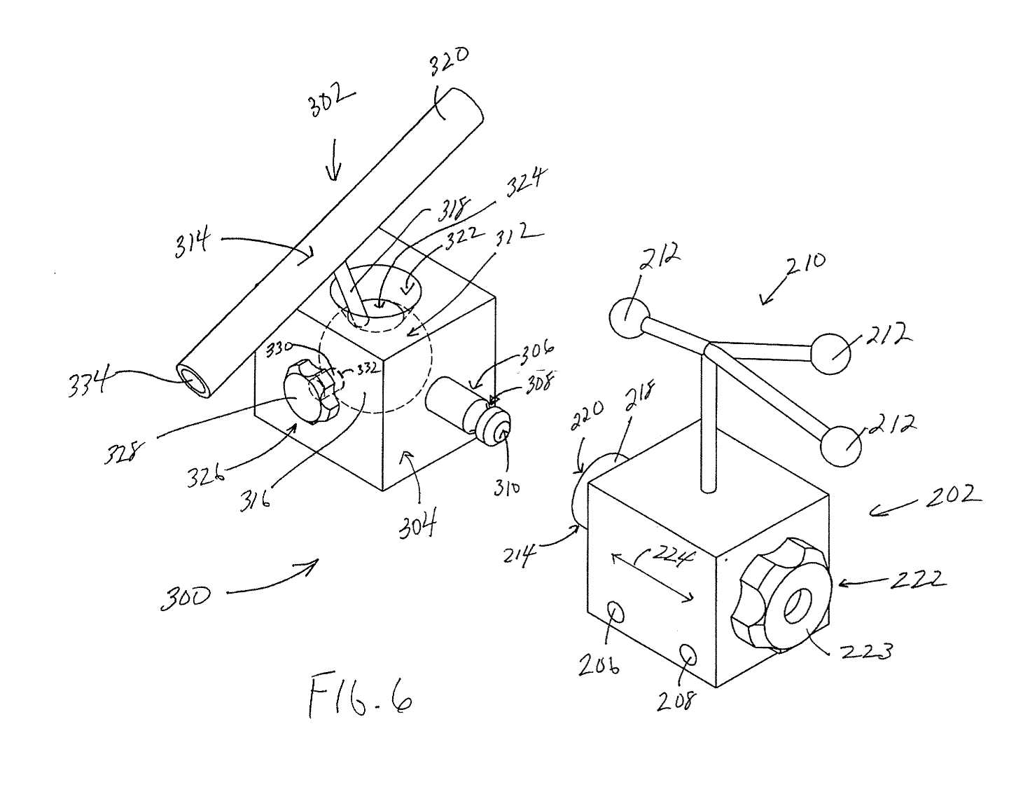

Referring now to FIG. 5, there is shown a surgical instrument 200 according to another embodiment of the present invention. The surgical instrument 200 includes a base or base member 202 having removably coupled thereto a surgical device 204, illustrated as being separated from the base 202. The surgical device is removably, adjustably and interchangeably coupled to the base member and can include a guide block. Two or more surgical devices can be used with the base member to provide modularity.

The device 204 is positionable with respect to the base 202. The base member 202 includes a first aperture 206 and a second aperture 208 each of which extends through the base member 202, such that screws, pins, or other attaching devices can be used to secure the surgical instrument 200 to a bone. The base member 202 includes a marker 210 which is removably attached to the base member 202. The marker 210 includes a plurality of reflectors 212 to enable the computer system to locate and/or track the base 202 with respect to a bone as well as with respect to the surgical device 204.

The surgical device 204 is removably coupled to the base member 202 which includes a fitting 214 which receives a press fit shaft 216 which extends from the surgical device 204. A press fit of the shaft 216 to the fitting 214 is illustrated in more detail in FIG. 7. The fitting 214 includes a body 218 having an aperture 220 which receives the shaft 216. The base member 202 includes a lock/positioner 222, here illustrated as a knurled knob 223, which can lock or hold the shaft 216 to the base 202 as well as to position the shaft 216 with respect to the base member 202. For instance, the lock/positioner 222 can be used to extend or to retract the surgical device 204 in a linear direction 224 by a mechanism contained within the base member 202 (not shown), as would be understood by one skilled in the art.

As stated, the surgical device 204 can be coupled to the base member 202 by insertion of the shaft 216 into the fitting 214. The surgical device 204 includes a second shaft 225 which is substantially perpendicular to the first shaft 216. The first shaft 216 is moveably coupled to the second shaft 225 through a positioner 226 having an aperture therethrough which receives the second shaft 225. A lock 228, which can include a knurled knob 229, enables the positioner 226 to be locked in place with respect to the second shaft 225. The positioner 226 can include a cylinder 230 having an aperture therethrough to receive the second shaft 224. The aperture of the cylinder 230 allows for sliding movement between the interior of the cylinder 230 and the second shaft 224 such that the surgical device 204 can be positioned with respect to the base member 202 in a rotational direction about an axis 232 and/or along a linear direction of a longitudinal axis 233. It is within the scope of the present invention for the movement to be only one of the rotational or longitudinal movements. Consequently, loosening of the lock 228 can enable the positioner 226 to rotate about the shaft 225 or along the direction 233, thereby allowing the surgical device 204 to be positioned as needed. The use of multiple positioners enable the device to move away from or closer to the base when fixed to the bone, enables the device to move up and down, or enables the angle of the device to be adjusted with respect to the base.

A second positioner 234, which can include a knurled knob 235, can be coupled to the second shaft 225 to extend through a first flange 236 and a second flange 238 positioned substantially parallel to the first flange 236. The positioner 234 enables the surgical device 204 to be positioned about rotational axis 239. The positioner 234 can include a knurled knob which can provide for frictional positioning of the surgical device 204 with respect to the base member 202 and for adjustment of the surgical device 204 about the rotational axis 239.

The surgical device 204 provides for substantially accurate alignment of a surgical tool with respect to the base member 202 as well as with respect to a main body 240 of the surgical device 204. For instance, the surgical device 204 includes a cutting guide 242 which can include a slot 244 having a length and a width selected to receive a surgical tool, such as a sawblade. The slot 244 extends from a first side of the body 246 through a second side of the body 248. While the positioner 222, positioner 234, and lock 228 can hold the device 204 in place with respect to the base member 202 to perform a surgical procedure, it is within the scope of the present invention to include a first aperture 250 and a second aperture 252 which extend through the body 240 to enable fixing the body 242 to a bone as would be understood by those skilled in the art.

The surgical device 204 can also include a marker 260 which can be removably coupled to the main body 240 as previously described with respect to the marker 210 coupled to the base member 202. The marker 260 includes a plurality of reflectors 262 which can be recognized by the computer navigational system to locate the surgical device 204 with respect to a bone. The marker 260, however, may not necessary, since it is within the scope of the present invention to use a surgical instrument having a marker incorporated therein. The marker on the surgical instrument can be used to locate the surgical instrument with respect to the surgical site.

In addition to the first cutting guide 242, a second cutting guide 264 includes a first rod 266 and a second rod 268 which extend from a rotatable guide platform 270. The first rod and second rod 266 and 268 respectively are spaced apart to form an alignment slot 272 which can receive a surgical instrument, such as a sawblade. The platform 270 can be coupled to the second shaft 225 whose position can be adjusted with the positioner 234. The platform 270 also includes a plurality of apertures 274 which can receive a pin, a screw or other known mechanism for fixing the cutting guide 264 with respect to a surgical site and to a bone.

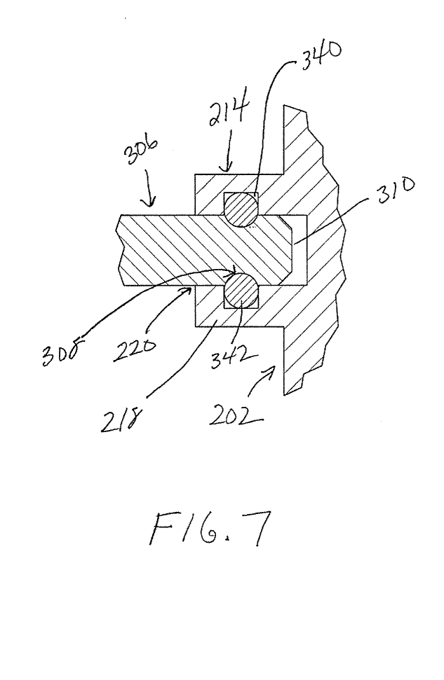

FIG. 6 illustrates another embodiment of a surgical instrument 300 which includes the base 202 as previously described and a second embodiment of a surgical device 302 coupled thereto. The surgical device 302 includes a base member 304 and a shaft 306 extending therefrom. The shaft 306 includes a locking feature including a channel 308 and an engaging nub 310 which fits within the fitting 214 of the base 202. The base member 304 further includes a substantially spherical aperture 312, or socket, formed within the base 304 which receives a drill guide 314. The drill guide 314 includes a sphere 316 having extending therefrom a rod or shaft 318 coupled to a drill locator 320.

The aperture 312 of the base member 304 is formed within the base member to include a wall 322 which extends from a substantially cylindrical aperture 324. The rod 318, which extends from the sphere 316, can therefore be positioned in any of a number of locations, the extent of which can be defined by the aperture 312, the sidewall 322, and the aperture 324. During a surgical procedure, the drill locator 320 can be positioned within the aperture 322 by loosening the lock positioner 326, here illustrated as including a knurled knob 328, coupled to a shaft 330. The shaft 330 extends through a threaded aperture in the base member 304 to enable an end portion 332 of the shaft 330 to contact the ball 316 to enable movement of and fixing the location of the drill locator 320 in position.

The drill locator 320 includes a hollow cylinder having a channel or aperture 334 which extends through the length of the cylinder. The channel 334 includes a diameter which can be determined based on the diameter of a selected drill passing through the drill locator 320. It is within the scope of the present invention for the drill locator 320 to be removably coupled to the sphere 316 such that a plurality of different drill locators can be included to enable a selected surgical technique to be performed with a required drill bit.

FIG. 7 illustrates a cross-sectional view of the fitting 214 of the base 202 which is coupled to the shaft 306 of the base 300 of FIG. 6. The illustration of FIG. 7, however, also applies to the surgical device 204 and the shaft 216 of FIG. 5 described herein. As illustrated, the shaft 306 extends into the aperture 220 of the fitting 214. The fitting 214 includes a channel or groove 340. Within the groove 340, a retaining ring 342 can be positioned to hold the shaft 306 to the base 202. The ring 342 contacts the channel or groove 308 of the shaft 306. The retaining ring 342 can include a compression fit ring such that insertion of the shaft 306 into the fitting 214 secures the shaft 306 and therefore the base member 304 to the base 202. It is also within the scope of the present invention to include bearings which are spring biased to the fitting 214 to provide a pressure fit of the device as would be understood of those skilled in the art.

While a preferred embodiment according to the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A surgical instrument for use with a surgical navigation system comprising:
a base adapted to be attached to a bone and having a first marker connected thereto, the first marker being trackable by the surgical navigation system; and
at least two surgical devices removably, adjustably and interchangeably coupled to the base member.

2. The surgical instrument of claim 1, wherein the base comprises a lock adapted to couple to the at least two surgical devices to fix the position of the surgical devices relative to the base.

3. The surgical instrument of claim 2, wherein the lock comprises a positioner adapted to position the surgical tools with respect to the base.

4. The surgical instrument of claim 1, wherein one of the surgical devices comprises a second marker connected thereto.

5. The surgical instrument of claim 1, wherein the surgical devices each comprise a body and a shaft coupled thereto, the shaft adapted to be removably coupled to the base.

6. The surgical instrument of claim 5, wherein the shaft comprises a lock.

7. The surgical instrument of claim 6, wherein the lock comprises a groove.

8. The surgical instrument of claim 1, wherein the surgical devices each comprise a body and a positioner adjustably connected to the body.

9. The surgical instrument of claim 8, wherein the positioner is adapted to move the body with respect to the base in at least one direction.

10. The surgical instrument of claim 9, wherein the positioner is adapted to move the device with respect to the positioner in a linear direction and in a rotational direction.

11. The surgical instrument of claim 8, wherein the body comprises a guide adapted to receive a tool.

12. A surgical apparatus comprising:
a base adapted to be coupled to a bone of a patient;
a first reference array coupled to the base;
a tool guide adjustably and removably coupled to the base; and
an adjustment mechanism, coupled to one of the base and the tool guide, the adjustment mechanism including a first positioner and a second positioner, the first positioner adapted to move the tool guide relative to the first reference array in a first direction, and the second positioner adapted to move the tool guide in a second direction different than the first direction.

13. The surgical apparatus of claim 12, wherein one of the first direction and the second direction comprises a rotational direction.

14. The surgical apparatus of claim 13, wherein the adjustment mechanism comprises a third positioner, the third positioner being adapted to move the tool guide in a third direction different than the first direction and the second direction.

15. The surgical apparatus of claim 14, wherein one of the first direction, second direction, and third direction comprises a rotational direction.

16. A method of cutting a bone during a surgery aided by a surgical navigation system, comprising:
mounting a base member having a reference array to a bone;
attaching a cutting guide to the reference array and then positioning the cutting guide using a tracked instrument;
fixing the position of the cutting guide relative to the base member; and
resecting the bone.

17. The method of claim 16, wherein the desired cutting location is displayed on a monitor.

18. The method of claim 16, wherein the resecting comprises inserting a saw blade into a cutting slot of the cutting guide.

19. The method of claim 16, wherein the resection is made to a femur.
